(19) United States
(12) Patent Application Publication
(10) Pub. No.: US 2012/0157891 A1
(43) Pub. Date: Jun. 21, 2012

(54) THERMAL AND PRESSURE WAVE TREATMENT

(76) Inventor: Moshe Ein-Gal, Ramat Hasharon (IL)

(21) Appl. No.: 12/972,669

(22) Filed: Dec. 20, 2010

(51) Int. Cl. A61N 7/02; A61N 7/00

(52) U.S. Cl. 601/3; 601/2

(57) ABSTRACT

A method including directing pressure waves at a tissue, and heating the tissue with thermal energy pulses, the thermal energy pulses synchronized to arrive at the tissue simultaneously with the pressure waves within a time tolerance range, and wherein heat of each thermal energy pulse is significantly dissipated in an environment that includes the tissue before a subsequent thermal energy pulse arrives at the tissue.

### FIELD OF THE INVENTION

The present invention generally relates to a system and method for treating tissue with a combination of thermal and pressure wave energy.

### BACKGROUND OF THE INVENTION

Tissue treatment by heating is used for diathermy, coagulation, surgery, hyperthermia, pain relief, drug delivery assistance, and many others. Heating tissue may be done by means of RF, ultrasound, laser light, electromagnetic induction, convection, mechanical stimulation and others.

Tissue treatment by non-heating pressure waves is used for urinary stones disintegration (lithotripsy), pain alleviation in joints, skin treatment, revascularization, massage and others. Non-heating pressure waves include sub-ultrasonic waves and shockwaves. Typical sub-ultrasonic waves are applied as pulses of sub-ultrasonic content and sub-ultrasonic repetition rate. Typical shockwaves have a steep wave front, followed by a shallower rarefaction tail that decays in oscillatory fashion. Extracorporeal shockwaves for medical applications are typically produced by electrohydraulic, electromagnetic or piezoelectric methods. Electrohydraulic shockwaves are formed with a high energy spark in water and an ellipsoidal reflector is used to focus the waves. Electromagnetic shockwaves are formed by producing a current pulse in a coil and inducing opposite current in an adjacent conducting membrane submerged in water. The repelling force of the opposing currents jerks the membrane and produces a wave. Focusing is by an acoustic lens, a reflector or by shaping a spherical membrane. Intracorporeal shockwaves are also used in lithotripsy, for example, and are produced by focusing laser light or creating a spark at the target.

### SUMMARY OF THE INVENTION

The present invention seeks to provide improved treatment modalities by combining thermal energy with pressure wave energy without causing significant temperature increase for extended time, as is described more in detail hereinbelow.

In accordance with a non-limiting embodiment of the invention, pulsed heating is synchronized with pulses of pressure waves such that both pulses reach the target simultaneously. In one example, the propagation speed of the wave may be about 1.5 m/msec, and the time of releasing the heating pulse depends on the propagation speed of the heating pulse and the respective distances of the thermal and wave devices to the target. The heat of each pulse is dissipated prior to the arrival of the subsequent heating pulse.

There is thus provided in accordance with a non-limiting embodiment of the present invention a method including directing pressure waves at a tissue, and heating the tissue with thermal energy pulses, the thermal energy pulses synchronized to arrive at the tissue simultaneously with the pressure waves within a time tolerance range, and wherein heat of each thermal energy pulse is significantly dissipated in an environment that includes the tissue before a subsequent thermal energy pulse arrives at the tissue. The deposited heat energy per pulse, the number of pulses and the repetition rate of the pulses are determined by a processor according to the temperature and the heat dissipation capability of the tissue.

According to an embodiment of the present invention, the tissue temperature is measured by a sensor in communication with the processor. The heat dissipation capacity of the tissue may be based on prior measurements of tissue properties or properties based on the assumption that the tissue is similar to previously published tissue properties; these properties include, but are not limited to, thermal conductivity, specific heat, coefficients of thermal convection (forced and free), and others, both for dry and wet tissues.

The pressure waves may include sub-ultrasonic pulses or shockwaves or a combination of both. The pressure waves may include extracorporeal or intracorporeal pressure waves or a combination of both. The thermal energy may include RF heating, ultrasonic heating, optical heating or electromagnetic induction heating or any combination thereof.

In accordance with an embodiment of the present invention the method includes focusing at least one of the pressure waves and the thermal energy pulses. Focusing is according to the shape of the treated tissue: the focal volume may include spherical, ellipsoidal-like or generally elongated shapes.

In accordance with an embodiment of the present invention the method includes localizing the target by producing images of the target and processing the images with respect to a reference to calculate a location of the target with respect to a coordinate system. Imaging may be done by ultrasound, x-ray, CT, MRI, optical or electrical imaging or any combination thereof.

There is also provided in accordance with a non-limiting embodiment of the present invention system including a pressure wave source for directing pressure waves at a tissue, a heat source for heating the tissue with thermal energy pulses, and a controller for synchronizing the thermal energy pulses to arrive at the tissue simultaneously with the pressure waves within a time tolerance range, and such that heat of each thermal energy pulse is dissipated in an environment neighboring the tissue before a subsequent thermal energy pulse arrives at the tissue.

In accordance with an embodiment of the present invention a focusing element focuses the pressure waves.

In accordance with an embodiment of the present invention a focusing element focuses the thermal energy pulses.

### BRIEF DESCRIPTION OF THE DRAWINGS

The present invention will be understood and appreciated more fully from the following detailed description, taken in conjunction with the drawings in which:

FIG. 1 is a simplified illustration of a system and method for treating tissue with a combination of thermal energy and pressure wave energy, in accordance with an embodiment of the present invention, using RF heating;

FIG. 2 is a simplified illustration of a system and method for treating tissue with a combination of thermal energy and pressure wave energy, in accordance with an embodiment of the present invention, using ultrasonic heating; and

FIG. 3 is a simplified flow chart of a method for treating tissue with a combination of thermal energy and pressure wave energy, in accordance with an embodiment of the present invention.

### DETAILED DESCRIPTION OF EMBODIMENTS

Reference is now made to FIG. 1, which illustrates system and method for treating tissue with a combination of thermal energy and pressure wave energy, in accordance with a non-limiting embodiment of the present invention.

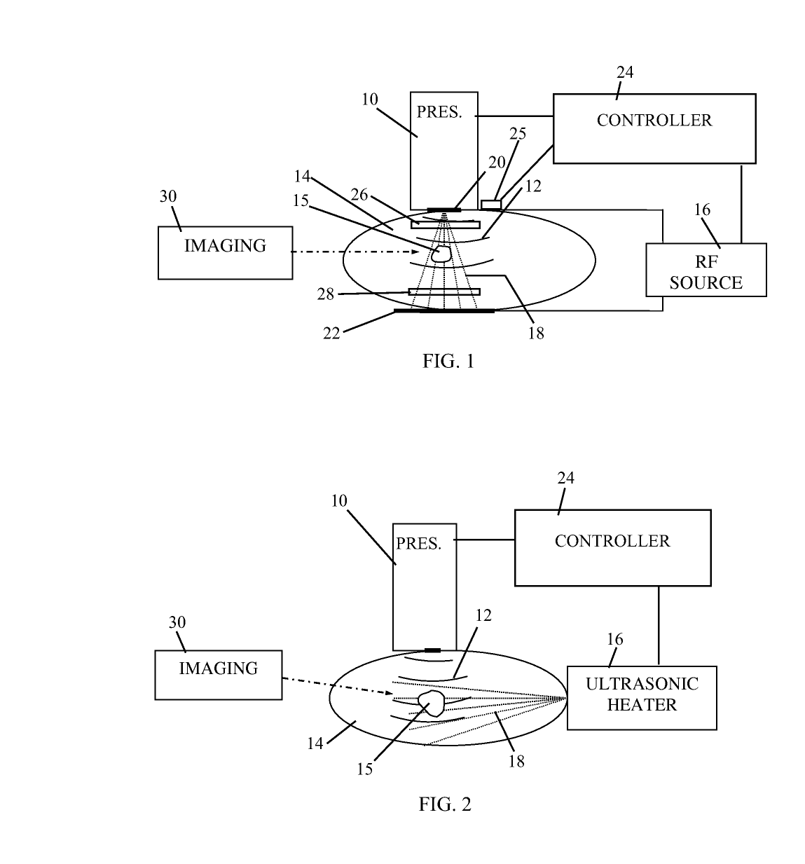

The system includes a pressure wave source 10 for directing pressure waves 12 at a tissue 14 (typically a target 15 in the tissue). The system also includes a heat source 16 for heating tissue 14 with thermal energy pulses 18. In the embodiment of FIG. 1, heat source 16 is a radio-frequency Joule (RF) heat source that includes an RF electrode 20 and a counter electrode 22 placed on opposite sides of tissue 14. In the embodiment of FIG. 2, heat source 16 is an ultrasonic heater and the thermal energy pulses 18 are delivered as ultrasonic waves. The invention is not limited to these types of heat sources, and the thermal energy may include RF heating, ultrasonic heating, optical heating or electromagnetic induction heating or any combination thereof.

The system includes a controller 24 for synchronizing the thermal energy pulses 18 to arrive at tissue 14 simultaneously with pressure waves 12 within a time tolerance range. The controller 24 controls the timing such that the heat of each thermal energy pulse 18 is dissipated in an environment neighboring tissue 14 before a subsequent thermal energy pulse 18 arrives at tissue 14. In accordance with one embodiment of the invention the time tolerance range is ±0.1 msec. In accordance with another embodiment of the invention the time tolerance range is ±0.5 msec. In accordance with yet another embodiment of the invention the time tolerance range is ±1 msec. Other ranges may be used, each having their own characteristics, advantages and tradeoffs, depending on the particular application.

The heat energy per pulse, the number of pulses and the repetition rate of the pulses are determined by controller 24 according to the temperature and the heat dissipation capability of the tissue. The tissue temperature can be measured by a sensor 25 in communication with controller 24. The heat dissipation capacity of the tissue may be based on prior measurements of tissue properties or properties based on the assumption that the tissue is similar to previously published tissue properties; these properties include, but are not limited to, thermal conductivity, specific heat, coefficients of thermal convection (forced and free), and others, both for dry and wet tissues

As seen in FIG. 1, one or more pressure wave focusing elements 26 may be provided for focusing the pressure waves 12, such as but not limited to, a focusing parabolic reflector, typically used in lithotripsy. One or more thermal energy focusing elements 28 maybe provided for focusing the thermal energy pulses 18, such as but not limited to, mirrors and/or lenses. In order to increase treatment efficiency while sparing surrounding tissue, the pressure waves and/or the heating are focused at the target. For example, focused shockwaves and focused heating ultrasound may deliver temporally simultaneous and spatially coinciding energy to the tissue in short pulses so as to keep the surrounding tissue unharmed.

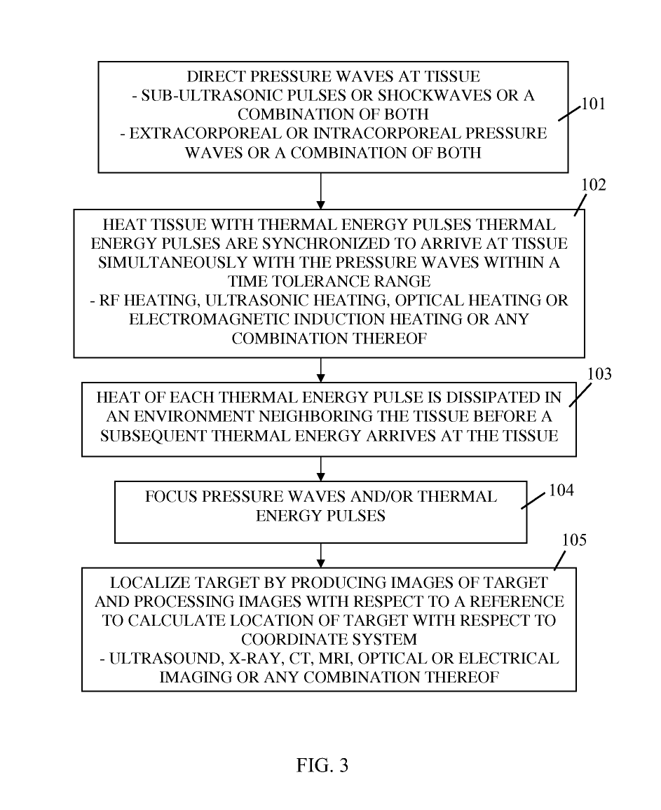

Reference is now made to FIG. 3, which is a simplified flow chart of a method for treating tissue with a combination of thermal energy and pressure wave energy, in accordance with an embodiment of the present invention.

The method includes directing pressure waves at a tissue (101), and heating the tissue with thermal energy pulses, wherein the thermal energy pulses are synchronized to arrive at the tissue simultaneously with the pressure waves within a time tolerance range (102). Heat of each thermal energy pulse is dissipated in an environment neighboring the tissue before a subsequent thermal energy pulse arrives at the tissue (103). The pressure waves may include sub-ultrasonic pulses or shockwaves or a combination of both. The pressure waves may include extracorporeal or intracorporeal pressure waves or a combination of both. The thermal energy may include RF heating, ultrasonic heating, optical heating or electromagnetic induction heating or any combination thereof.

The method further includes focusing at least one of the pressure waves and the thermal energy pulses (104).

The target may be localized by producing images of the target and processing the images with respect to a reference (e.g., an inertial reference frame of a coordinate system) to calculate a location of the target with respect to the coordinate system (105). Imaging may be done by ultrasound, x-ray, CT, MRI, optical or electrical imaging or any combination thereof (imaging system 30 shown in FIGS. 1 and 2).

The scope of the present invention includes both combinations and subcombinations of the features described hereinabove as well as modifications and variations thereof which would occur to a person of skill in the art upon reading the foregoing description and which are not in the prior art.

What is claimed is:

1. A method comprising:
directing pressure waves at a tissue; and
heating said tissue with thermal energy pulses, said thermal energy pulses synchronized to arrive at said tissue simultaneously with said pressure waves within a time tolerance range, and wherein heat of each thermal energy pulse is significantly dissipated in an environment that includes said tissue before a subsequent thermal energy pulse arrives at said tissue.

2. The method according to claim 1, wherein said pressure waves comprise at least one of sub-ultrasonic pulses and shockwaves.

3. The method according to claim 1, wherein said pressure waves comprise at least one of extracorporeal and intracorporeal pressure waves.

4. The method according to claim 1, wherein said thermal energy comprises at least one of RF heating, ultrasonic heating, optical heating and electromagnetic induction heating.

5. The method according to claim 1, comprising focusing at least one of said pressure waves and said thermal energy pulses.

6. The method according to claim 1, the thermal energy per pulse, number of pulses and repetition rate of said pulses are determined according to a temperature and heat dissipation capability of the tissue.

7. The method according to claim 1, further comprising localizing said target by producing images of said target and processing said images with respect to a reference to calculate a location of said target with respect to a coordinate system.

8. The method according to claim 7, comprising producing said images with at least one of ultrasound, x-ray, CT, MRI, optical and electrical imaging.

9. The method according to claim 1, wherein said time tolerance range is ±0.1 msec.

10. The method according to claim 1, wherein said time tolerance range is ±0.5 msec.

11. The method according to claim 1, wherein said time tolerance range is ±1 msec.

12. A system comprising:
a pressure wave source for directing pressure waves at a tissue;
a heat source for heating said tissue with thermal energy pulses; and
a controller for synchronizing said thermal energy pulses to arrive at said tissue simultaneously with said pressure waves within a time tolerance range, and such that heat of each pulse of thermal energy is dissipated in an environment neighboring said tissue before a subsequent pulse of thermal energy arrives at said tissue.

13. The system according to claim 12, wherein said pressure waves comprise at least one of sub-ultrasonic pulses and shockwaves.

14. The system according to claim 12, wherein said thermal energy comprises at least one of RF heating, ultrasonic heating, optical heating and electromagnetic induction heating.

15. The system according to claim 12, comprising a focusing element for focusing said pressure waves.

16. The system according to claim 12, comprising a focusing element for focusing said thermal energy pulses.

17. The system according to claim 12, further comprising a sensor in communication with said controller for measuring tissue temperature, and wherein said controller controls the thermal energy per pulse, number of pulses and repetition rate of said pulses according to a temperature and heat dissipation capability of the tissue.
